(19) United States
(12) Patent Application Publication
(10) Pub. No.: US 2021/0283207 A1
(43) Pub. Date: Sep. 16, 2021

(54) ADIPONECTIN SECRETION PROMOTING AGENT

(71) Applicants: Hirosuke OKU, Okinawa (JP); OSAKA UNIVERSITY, Osaka (JP); Tokunoshima Town, Kagoshima (JP); NPO AMAMI FUNCTIONAL FOODS STUDY GROUP, Kagoshima (JP)

(72) Inventors: Hirosuke OKU, Okinawa (JP); Satoshi OHNO, Osaka (JP); Miki SAKAUE, Osaka (JP); Shojiro FUJIYAMA, Kagoshima (JP); Yasuo KAMIYAMA, Osaka (JP)

(73) Assignees: OSAKA UNIVERSITY, Osaka (JP); Tokunoshima Town, Kagoshima (JP); Hirosuke OKU, Okinawa (JP); NPO AMAMI FUNCTIONAL FOODS STUDY GROUP, Kagoshima (JP)

(21) Appl. No.: 17/261,377

(22) PCT Filed: Jul. 17, 2019
(86) PCT No.: PCT/JP2019/028166

§ 371 Date: Jan. 19, 2021

(30) Foreign Application Priority Data
Jul. 19, 2018 (JP) 2018-136131

(51) Int. Cl. A61K 36/28; A61K 9/00; A23L 33/00; A23L 33/105; A23L 2/52

(52) U.S. Cl. CPC A61K 36/28; A61K 9/0056; A23V 2002/00; A23L 33/105; A23L 2/52; A23L 33/40

(57) ABSTRACT

The present invention provides an adiponectin secretion promoting agent comprising Cirsium brevicaule A. Gray, preferably a dry powder of the leaves of Cirsium brevicaule A. Gray, and also provides a food or drink for promoting adiponectin secretion comprising the same.

### TECHNICAL FIELD

The present invention relates to an adiponectin secretion promoting agent.

### BACKGROUND ART

Cirsium brevicaule A. Gray (CBAG) is a plant belonging to the genus Cirsium in the family Asteraceae. CBAG grows wild largely in the coastal areas of the islands south of Kagoshima Prefecture in Japan. CBAG is traditionally used as not only a food stuff but also as herbal medicine in the Okinawa Islands and Amami Islands of Japan. The inventors have reported that administration of a CBAG powder to mice fed a high-fat diet reduced the blood concentration of fatty acids, reduced the amount of subcutaneous fat, reduced the amount of liver fat, and inhibited the expression of fatty acid synthase (patent literature 1 and non-patent literature 1).

Adiponectin is a cytokine (adipokine) secreted from adipocytes, and is known as a good adipokine. Adiponectin is also known to be closely associated with lifestyle-related diseases, such as circulatory system diseases, diabetes mellitus and obesity. Type 2 diabetic model mice with high-fat diet loading develop adipocyte hypertrophy and exacerbation of insulin resistance, and show significant decrease in the adiponectin concentration in the blood. However, replenishment of adiponectin in obese type 2 diabetic model mice with high-fat diet loading ameliorates insulin resistance (non-patent literature 2). Adiponectin gene-deficient mice exhibit various signs of metabolic syndrome, including insulin resistance, abnormal glucose tolerance, abnormal lipid metabolism and hypertension. Therefore decrease in adiponectin levels due to obesity may at least partially contribute to impaired glucose tolerance, abnormal lipid metabolism and hypertension.

### CITATION LIST

### Patent Literature

Patent literature 1: WO 2015/022978

### Non-Patent Literature

Non-patent literature 1: Inafuku M, et al. Cirsium brevicaule A. GRAY leaf inhibits adipogenesis in 3T3-L1 cells and C57BL/6 mice, Lipids Health Dis. 2013 Aug. 15; 12:124.

Non-patent literature 2: Yamauchi T, et al., The fat-derived hormone adiponectin reverses insulin resistance associated with both lipoatrophy and obesity, Nature med 7: 941-946, 2001.

### SUMMARY OF INVENTION

### Technical Problem

An object of the present invention is to provide a novel adiponectin secretion promoting agent that is safe and low toxic and can be administered for a long period of time.

### Solution to Problem

The present invention was made to solve the above problems and includes the following.
[1] An adiponectin secretion promoting agent comprising Cirsium brevicaule A. Gray.
[2] The adiponectin secretion promoting agent according to the above [1], wherein the Cirsium brevicaule A. Gray is in the form of a dry powder.
[3] A food or drink for promoting adiponectin secretion, comprising the adiponectin secretion promoting agent according to the above [1] or [2].

### Advantageous Effects of Invention

The present invention provides an adiponectin secretion promoting agent that is safe, low toxic and effective in humans and can be administered for a long period of time.

### DESCRIPTION OF EMBODIMENTS

The inventors conducted clinical study to examine the impact of Cirsium brevicaule A. Gray on lipid metabolism in humans using 24 subjects of Japanese adult men and women with borderline to moderately high levels of LDL cholesterol (120 to 159 mg/dL) or normally high to moderately high levels of triglyceride (120 to 199 mg/dL). The subjects ingested a dry powder of Cirsium brevicaule A. Gray three times a day (3 g×3/day) for 12 weeks, and the inventors found that the adiponectin concentration in the blood significantly increased at 8 and 12 weeks after the ingestion. The results indicate that Cirsium brevicaule A. Gray is useful as an active ingredient of an adiponectin secretion promoting agent.

The present invention provides an adiponectin secretion promoting agent comprising Cirsium brevicaule A. Gray. The

Cirsium brevicaule A. Gray may be those harvested after any days of cultivation. The part used of Cirsium brevicaule A. Gray is not particularly limited, and may be any one of the leaves, stems, roots, rhizomes, fruits, seeds, seed coats, flowers, etc. of Cirsium brevicaule A. Gray. Preferred are the leaves of Cirsium brevicaule A. Gray.

Cirsium brevicaule A. Gray used in the adiponectin secretion promoting agent of the present invention may be fresh or dried one. Cirsium brevicaule A. Gray used in the adiponectin secretion promoting agent may be in the form of a dry powder, a lyophilized powder, a juice, an extract, etc. Cirsium brevicaule A. Gray may be dried by a known drying technique, for example, natural drying, hot air (warm air) drying, cold air drying, reduced pressure drying, lyophilization, etc. After dried, Cirsium brevicaule A. Gray may be powdered by, for example, pulverizing it in a commercially available mill and then removing non-powdered Cirsium brevicaule A. Gray. A juice of Cirsium brevicaule A. Gray may be produced with a commercially available juice machine. An extract of Cirsium brevicaule A. Gray may be produced by subjecting Cirsium brevicaule A. Gray to extraction process in a solvent. The Cirsium brevicaule A. Gray to be subjected to extraction process may be fresh Cirsium brevicaule A. Gray, minced fresh Cirsium brevicaule A. Gray, pulverized fresh Cirsium brevicaule A. Gray, or dried Cirsium brevicaule A. Gray, pulverized dried Cirsium brevicaule A. Gray, or powdered dried Cirsium brevicaule A. Gray.

The solvent for the extraction may be water, alcohols, hexane, chloroform, ethers, esters or ketones. Examples of the alcohols include, for example, ethanol, methanol, n-propanol, isopropanol, n-butanol, 1,3-butylene glycol, propylene glycol, and glycerol. Examples of the ethers include, for example, diethyl ether, and propyl ether. Examples of the esters include, for example, butyl acetate, and ethyl acetate. Examples of the ketones include, for example, acetone, and ethyl methyl ketone. A mixture of two or three or more of these solvents may also be used. Different solvents may be sequentially used in extraction process. For example, extraction process may be performed in hexane, and the resulting residue is subjected to extraction process in chloroform.

The Cirsium brevicaule A. Gray extract may be in the form of an extract liquid, a concentrated or diluted extract liquid, a solid produced from an extract liquid, or a dry powder produced from an extract liquid.

The adiponectin secretion promoting agent of the present invention may comprise a dry powder of Cirsium brevicaule A. Gray, a lyophilized powder of Cirsium brevicaule A. Gray, a dry powder of the leaves of Cirsium brevicaule A. Gray, or a lyophilized powder of the leaves of Cirsium brevicaule A. Gray. Such dry powders and lyophilized powders can be produced by a known method. For example, a lyophilized powder of the leaves of Cirsium brevicaule A. Gray may be produced by the method as described in the Examples.

Since the adiponectin secretion promoting agent of the present invention significantly increases the adiponectin concentration in the blood, the adiponectin secretion promoting agent can be suitable as a medicine or food or drink for improving insulin resistance, for which replenishment of adiponectin is known to be effective (non-patent literature 2), or as a medicine or food or drink for alleviating hepatopathy (Fukushima J, et al., Hepatol Res. 2009 July ; 39 (7) :724-738), or as a medicine or food or drink for inhibiting liver fibrosis (Kamada Y et al., Gastroenterology. 2003 December ; 125 (6):1796-807).

The adiponectin secretion promoting agent of the present invention can also be used to treat or improve hypoadiponectinemia. The term “hypoadiponectinemia” as used herein refers to health conditions that are expected to be improved by promotion of adiponectin secretion in the body.

The adiponectin secretion promoting agent of the present invention can also be used to treat or improve conditions and diseases caused by hypoadiponectinemia. For example, the adiponectin secretion promoting agent can be used to treat or improve metabolic syndrome, cancers associated with lifestyle-related diseases, insulin resistance syndrome, diabetes mellitus (including type I diabetes mellitus and type II diabetes mellitus), diabetic complications (including retinopathy, renal dysfunction, neurosis, cataract, coronary artery diseases, etc.), arteriosclerosis, coronary artery diseases, renal dysfunction, myocardial infarction, hypertension, cerebrovascular accident, hyperlipemia, hypercholesterolemia, obesity, bone density reduction, liver diseases, etc. The adiponectin secretion promoting agent of the present invention can also be used for anti-aging.

The adiponectin secretion promoting agent of the present invention can be embodied in the form of a medicine. The medicine of the present invention comprises Cirsium brevicaule A. Gray as an active ingredient, and can be formulated according to a conventional method. Examples of such a formulation for oral administration include solid or liquid dosage forms, and specific examples thereof include tablets (including sugar-coated tablets and film-coated tablets), pills, granules, powders, capsules (including soft capsules), syrups, emulsions, and suspensions. These formulations are produced by a known method, and contain carriers, diluents or excipients that are typically used in the pharmaceutical field. Examples of carriers and excipients for tablets include lactose, starch, sucrose, and magnesium stearate. Examples of a formulation for parenteral administration include injections and suppositories. Injections include intravenous injection, subcutaneous injection, intradermal injection, intramuscular injection, intravenous drip infusion, and intraarticular injection. Such injections can be prepared by a known method. For example, the injections can be prepared by dissolving, suspending or emulsifying the active ingredient in an aseptic aqueous or oily liquid typically used for preparation of injections. The aqueous liquid for injection may be, for example, physiological saline and an isotonic solution containing glucose or other auxiliary agents, and may be used in combination with an appropriate solubilizing agent, such as alcohols (e.g., ethanol etc.), polyalcohols (e.g., propylene glycol, polyethylene glycol, etc.), and nonionic surfactants (e.g., polysorbate 80, HCO-50, etc.). The oily liquid may be, for example, sesame oil, soybean oil, etc., and may used in combination with a solubilizing agent, such as benzyl benzoate and benzyl alcohol. The suppositories used for rectal administration are prepared by mixing the active ingredient with a base typically used for preparation of suppositories.

The adiponectin secretion promoting agent of the present invention can also be embodied in the form of a food or drink. The food or drink includes health foods, functional foods, foods for specified health uses, foods for medical uses, and supplemental foods (dietary supplements). The form of the food or drink is not particularly limited. Examples of the form of the food or drink include drinks such as tea drink, refreshing drink, carbonated drink, nutritional drink, fruit juice, and lactic drink; noodles such as buckwheat noodle, wheat noodle, Chinese noodle, and instant noodle; sweets and bakery products such as drop, candy, chewing gum, chocolate, snack, biscuit, jelly, jam, cream, pastry, and bread; fishery and livestock products, such as fish sausage, ham, and sausage; dairy products such as processed milk and fermented milk; fats, oils and processed foods thereof, such as vegetable oil, oil for deep frying, margarine, mayonnaise, shortening, whipped cream, and dressing; seasonings such as sauce and dipping sauce; retort pouch foods such as curry, stew, rice-bowl cuisine, porridge, and rice soup; and frozen desserts, such as ice cream, sherbet, and shaved ice. The supplemental foods (dietary supplements) may be provided in the form of, for example, a tablet, granules, a powder, an energy drink, or the like.

The medicine for improving insulin resistance containing the adiponectin secretion promoting agent of the present invention or the food or drink for promoting adiponectin secretion containing the adiponectin secretion promoting agent of the present invention comprises as an active ingredient Cirsium brevicaule A. Gray, which is traditionally used as a food stuff. The medicine or the food or drink is therefore safe for humans and non-human mammals (e.g., rats, mice, rabbits, sheep, pigs, cows, cats, dogs, monkeys, etc.).

The dosage of the medicine or the intake of the food or drink can be determined depending on the age and body weight of a patient or an individual who ingests the medicine or the food or drink, the symptoms, the administration time, the dosage form, the mode of administration, a combination of drugs, etc. For example, when the adiponectin secretion promoting agent of the present invention is orally administered as a medicine, the daily dosage of a dry powder of Cirsium brevicaule A. Gray for an adult human may be 0.1 g or more, 0.2 g or more, 0.3 g or more, 0.4 g or more, 0.5 g or more, 0.6 g or more, 0.7 g or more, 0.8 g or more, 0.9 g or more, or 1.0 g or more, or may be 20 g or less, 18 g or less, 16 g or less, 15 g or less, 14 g or less, 13 g or less, 12 g or less, 11 g or less, or 10 g or less. The dosage may be divided and administered a plurality of times (e.g., 3 times) per day.

When the adiponectin secretion promoting agent of the present invention is ingested as a food or drink, the agent may be blended into a food or drink such that the daily intake of a dry powder of Cirsium brevicaule A. Gray for an adult human is 0.1 g or more, 0.2 g or more, 0.3 g or more, 0.4 g or more, 0.5 g or more, 0.6 g or more, 0.7 g or more, 0.8 g or more, 0.9 g or more, or 1.0 g or more, or is 20 g or less, 18 g or less, 16 g or less, 15 g or less, 14 g or less, 13 g or less, 12 g or less, 11 g or less, or 10 g or less. The intake may be divided and ingested a plurality of times (e.g., 3 times) per day.

### EXAMPLES

The present invention will be described in more detail below with reference to Examples, but the present invention is not limited thereto.

### Example 1

### Impact of Cirsium Brevicaule A. Gray on Lipid Metabolism in Humans

### Test Method

(1) Test Subjects

Twenty-four test subjects were selected from Japanese men and women who were 20 to 80 years old at the time of acquisition of informed consent and had borderline to moderate levels of LDL cholesterol (120 to 159 mg/dL) or normally high to moderately high levels of triglyceride (120 to 199 mg/dL).
(2) Preparation and Ingestion of Cirsium Brevicaule A. Gray

Leaves of Cirsium brevicaule A. Gray (CBAG) were washed with water, and sterilized with hypochlorous acid. After the water was thoroughly drained off, the leaves were subjected to pre-freezing. The CBAG leaves were then lyophilized with a vacuum dryer (Marui & Co., Ltd.). The lyophilized leaves were crushed in a mill, and sieved to remove non-powdered leaves to give a lyophilized powder of the CBAG leaves. The test subjects ingested 3 g of the CBAG powder per dose, three times a day (9 g/day) for 12 weeks. Specifically, CBAG green juice for each dose was prepared at the time of use by adding 3 g of the CBAG powder to water (100 to 150 mL), and the test subjects ingested the CBAG green juice.
(3) Measurement Items
Body measurement: height (only for the initial measurement), body weight and body fat percentage.
General biochemical test: glucose, HbA1c, total cholesterol, HDL cholesterol, LDL cholesterol and triglyceride.
Adiponectin in blood.
Free fatty acids.

BMI was calculated by the following formula: BMI=body weight (kg)/[height (m)]².

Glucose, HbA1c, total cholesterol, HDL cholesterol, LDL cholesterol and triglyceride were measured at LSI Medience Corporation.

Adiponectin was measured using Human Adiponectin ELISA kit (Otsuka Pharmaceutical).

Free fatty acids were measured using NEFA C-Test Wako (Wako Pure Chemical Industries).
(4) Test Schedule

Body measurement was performed and the blood (8 mL) was collected for measurement of each item before the start of ingestion of the CBAG green juice and 4, 8 and 12 weeks after ingestion of the CBAG green juice.

### Results

The measured values at each time point were compared with those measured before the start of ingestion as a reference by paired t-test. The results are shown in Table 1 (Mean±standard deviation). No changes were observed for the body weight, BMI, and the triglyceride, free fatty acid, and fasting blood glucose levels. The body fat percentage significantly increased at 8 and 12 weeks after the start of the ingestion. The total cholesterol levels significantly increased at 12 weeks after the start of the ingestion. The HDL cholesterol levels significantly increased at 4, 8 and 12 weeks after the start of the ingestion. The LDL cholesterol levels significantly increased at 8 and 12 weeks after the start of the ingestion. The HbA1c levels significantly increased at 4 and 12 weeks after the start of the ingestion. The adiponectin levels significantly increased at 8 and 12 weeks after the start of the ingestion.

TABLE 1

| n = 24 | Before ingestion | 4 weeks later | 8 weeks later | 12 weeks later |
|---|---|---|---|---|
| Age | 51.2 ± 8.8 | | | |
| Sex (men/women) | 4/20 | | | |
| Body weight (kg) | 57.0 ± 9.7 | 56.9 ± 9.6 | 57.2 ± 9.7 | 57.2 ± 9.7 |
| BMI | 21.8 ± 3.1 | 21.8 ± 3.1 | 21.9 ± 3.2 | 21.9 ± 3.2 |
| Body fat percentage (%) | 26.6 ± 7.3 | 26.9 ± 7.4 | 27.4 ± 7.1* | 27.4 ± 7.3** |
| Total cholesterol (mg/dl) | 231.2 ± 24.0 | 227.9 ± 26.1 | 236.3 ± 20.7 | 241.3 ± 22.2* |
| HDL cholesterol (mg/dl) | 62.3 ± 13.7 | 64.6 ± 13.1* | 67.2 ± 14.2** | 67.7 ± 14.4** |
| LDL cholesterol (mg/dl) | 140.2 ± 16.8 | 136.5 ± 22.9 | 146.6 ± 15.8* | 149.0 ± 18.0** |
| Triglyceride (mg/dl) | 106.5 ± 58.6 | 110.1 ± 52.5 | 95.8 ± 37.1 | 97.0 ± 48.7 |
| Free fatty acids (mEq/l) | 0.76 ± 0.04 | 0.81 ± 0.05 | 0.83 ± 0.05 | 0.76 ± 0.04 |
| Fasting blood glucose | 81.8 ± 6.0 | 82.3 ± 4.8 | 81.5 ± 6.3 | 82.5 ± 5.8 |
| level (mg/dl) | | | | |
| HbA1c (%) | 5.3 ± 0.2 | 5.5 ± 0.2** | 5.4 ± 0.2 | 5.4 ± 0.2* |
| Adiponectin (ng/ml) | 12.3 ± 1.0 | 12.2 ± 1.3 | 13.2 ± 1.4* | 13.8 ± 1.6* |

*p < 0.05 vs before ingestion, **p < 0.01 vs before ingestion.

The present invention is not limited to each of the embodiments and Examples as described above, and various modifications are possible within the scope of the claims. Embodiments obtainable by appropriately combining the technical means disclosed in the different embodiments of the present invention are also included in the technical scope of the present invention.

1. An adiponectin secretion promoting agent comprising Cirsium brevicaule A. Gray.

2. The adiponectin secretion promoting agent according to claim 1, wherein the Cirsium brevicaule A. Gray is in the form of a dry powder.

3. A food or drink for promoting adiponectin secretion, comprising the adiponectin secretion promoting agent according to claim 1.

4. A food or drink for promoting adiponectin secretion, comprising the adiponectin secretion promoting agent according to claim 2.
